(19) United States
(12) Patent Application Publication
(10) Pub. No.: US 2007/0038139 A1
(43) Pub. Date: Feb. 15, 2007

(54) Chest tube air counters and methods

(76) Inventors: Anil K. Sinha, Lake Jackson, TX (US); Daniel DeOliveira, Lake Jackson, TX (US)

Correspondence Address: OSHA LIANG L.L.P., 1221 MCKINNEY STREET, SUITE 2800, HOUSTON, TX, 77010, US

(21) Appl. No.: 11/503,639

(22) Filed: Aug. 14, 2006

Related U.S. Application Data
(60) Provisional application No. 60/707,606, filed on Aug. 12, 2005.
(60) Provisional application No. 60/707,661, filed on Aug. 12, 2005.

(51) Int. Cl. A61B 5/08

(52) U.S. Cl. 600/538; 600/539

(57) ABSTRACT

An air counter for use with a chest tube includes a housing having an inlet and an outlet, at least one of which is configured to connect with the chest tube; a gas flow detection mechanism; and an electronic module having a memory for storing gas flow records. A method for draining a pleural cavity with a chest tube includes inserting a chest tube into the pleural cavity; and connecting an air counter with the chest tube, wherein the air counter comprises: a housing having an inlet and an outlet, at least one of which is configured to connect with the chest tube; a gas flow detection mechanism; and an electronic module having a memory for storing gas flow records.

### CROSS REFERENCE TO RELATED APPLICATIONS

The application claims the benefits of Provisional Application Ser. No. 60/707,606, filed on Aug. 12, 2005, entitled “Air Counter,” which is incorporated by reference in its entirety. In addition, this application is related to the following two applications filed on the same date: “Self Securing Chest Tubes and Methods,” and “Apparatus and Methods for Safe and Efficient Placement of Chest Tubes.” These two related applications are also incorporated by reference in their entireties.

### BACKGROUND OF INVENTION

1. Field of the Invention

The invention relates generally to chest tubes used in draining the pleural cavity. Particularly, it related to devices and methods for counting and monitoring the quantities of air/gas drainage in a chest tube system.

2. Background Art

Our lungs are protected by 24 ribs, which also shape and support the chest wall. The parietal pleura is a membrane lining the chest cavity. The visceral pleura is a membrane lining the lungs. The space between these two membranes is known as the pleural space or pleural cavity. This space has a thin film of fluid to provide lubrication and cohesion between the parietal and visceral pleura. A healthy adult has approximately 20 to 25 mL of fluid in the pleural space. Normally, the drainage of this space is regulated by the lymphatic circulation.

Pleural effusion is the accumulation of pathological quantities of fluid in the pleural space. Excess pleural fluid may be caused by liver or kidney failure, congestive heart failure, infection or malignancy blocking the lymphatic system. Four types of fluids may accumulate in the pleural space: blood (hemothorax), serous fluid (hydrothorax), chyle (chylothorax), and pus (pyothorax or empyema). In addition, pneumothorax (collapsed lung) may be caused by trauma or other pathological conditions.

Each of these conditions generally requires the placement of a chest tube to drain the air or fluids placing the patient in danger. The chest tube is a flexible tube that is inserted through the side of the chest into the pleural space. It is used to remove air, fluid, or pus from the pleural space. The free end of the tube is usually attached to an underwater seal, below the level of the chest. This allows air or fluid to escape from the pleural space and prevents anything from returning to the chest. Alternatively, the tube can be attached to a flutter valve to allow patients more mobility.

Chest tubes are usually inserted under local anesthesia. The skin over the area of insertion is first cleansed with antiseptic solution before sterile drapes are placed around the area. The local anesthetic is injected into the skin and down to the muscle. After the area is numb, a passage is made through the skin and muscle into the chest. This is often carried out by performing an incision in the skin with a scalpel blade. Then, the surgeons will use a Kelly clamp to perform blunt dissection of the soft tissues. There is no depth control in the Kelly clamp which may inadvertently go too far inside the pleural cavity. Once the tube is in place it is stitched to the skin to prevent it falling out and a dressing applied to the area. The tube stays in for as long as there is air or fluid to be removed or as long as there is risk of air gathering.

While fluid drainage is more readily measurable, air drainage is more difficult to assess. The information on the exact amount of air that has leaked through a chest tube system in a specified time period is critical in deciding when to remove a chest tube from a patient. Accordingly, there exists a need for devices and methods that can help doctors decide the history and status of air drainage via the chest tubes.

### SUMMARY OF INVENTION

One aspect of the invention relates to air counters for use with chest tubes. An air counter in accordance with one embodiment of the invention includes a housing having an inlet and an outlet, at least one of which is configured to connect with the chest tube; a gas flow detection mechanism; and an electronic module having a memory for storing gas flow records.

Another aspect of the invention relates to methods for draining a pleural cavity with a chest tube. A method in accordance with one embodiment of the invention includes inserting a chest tube into the pleural cavity; and connecting an air counter with the chest tube, wherein the air counter comprises: a housing having an inlet and an outlet, at least one of which is configured to connect with the chest tube; a gas flow detection mechanism; and an electronic module having a memory for storing gas flow records.

Other aspects and advantages of the invention will be apparent from the following description and the appended claims.

### BRIEF DESCRIPTION OF DRAWINGS

FIG. 1 shows an air counter in accordance with one embodiment of the invention.

### DETAILED DESCRIPTION

Embodiments of the present invention relate to air counters for use with chest tubes. In another aspect, embodiments of the present invention relate to methods for using the air counters with chest tubes to monitor and/or quantify the amount of air drainage from pleural cavities.

Current management of a chest tube is based on dogmatic and imprecise information. In a typical process, the clinicians ask the patients to cough in order to assess air leak in the Pleurovac system. If a patient has an intermittent air leak, the air leak may be missed at the exact moment of the clinician's evaluation. This can result in premature removal of a chest tube and recurrence of Pneumothorax.

Air Counters in accordance with embodiments of the invention allow for accurate measurement of unidirectional flow of air in a chest tube system. These air counters can precisely measure an exact volume of air to allow the clinician to know the exact amount of air that has leaked through a chest tube system in a specified time period. This information is critical in deciding when to remove a chest tube from a patient. Proper removal of a chest tube can prevent recurrence of Pneumothorax.

In accordance with embodiments of the invention, various mass flow measurement mechanisms may be used in the air counters. For example, some embodiments of the invention may use a turbine or a peddle wheel mechanism. Turbine flow meters measure the gas flow in a line via a rotor that spins as the gas passes through its blades. Paddle wheel flow meters have a paddle wheel that is perpendicular to the flow path, not parallel as in turbine flow meters. While these are the more common types of flow measurement systems, one of ordinary skill in the art would appreciate that air counters in accordance with embodiments of the invention are not limited to these types.

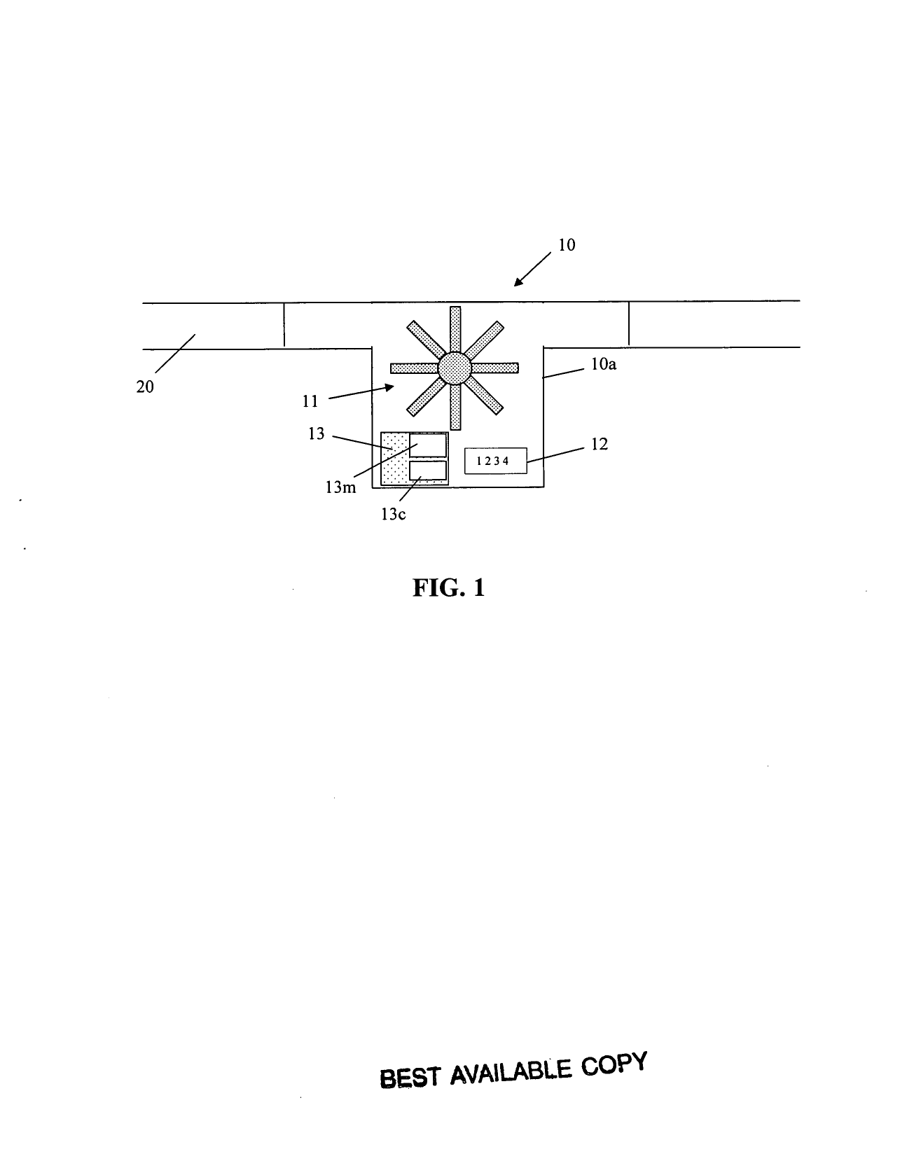

FIG. 1 shows an air counter in accordance with one embodiment of the invention. As shown, an air counter 10 is attached in-line to a chest tube 20 (or a branch of a chest tube). The air counter 10 may be housed in a housing 10a made of a suitable material such as plastic, composite, polymers, metal, glass, etc. The air counter 10 may have one or two connector ends adapted for connection with a chest tube 20, which may comprise a single tube for both liquid and air drainage or have separate branches (e.g., a Y-shape configuration) to allow liquid and air to drain off different branches.

The air counter 10 in the example of FIG. 1 includes a peddle wheel 11 that will rotate as the air passes the air counter. Rotation of the peddle wheel 11 is used to assess the amount of air passing the air counter 10. The air counter 10 and the chest tube 20 set up, as shown in FIG. 1, may only allow air to pass in one direction such that the air can only leak out from the pleural space, but not from outside into the pleural space. Such unidirectional flow may be accomplished by having the air counter 10 include the one-way flow capability, such as allowing the peddle wheel to turn in one direction only. Alternatively, the set up may include a one-way valve (not shown) that is not part of the air counter 10. The use of one-way valve, such as a Heimlich valve, is well known in the art.

Heimlich valve is small, light weight one-way valve contained in a clear, hard plastic shell. The valve consists of one or more rubber flutter leaflets that are compressed at the distal end. These leaflets allow one-way flow such that air, fluid, or blood clots exiting through the valve can't return to the pleural space. Heimlich valve is attached to connecting tubing, which is in turn attached to patient's chest tube.

The amount of air passing by the air counter 10 is recorded. This recorded information is stored in a memory and/or displayed to the user (patients or physicians). Thus, the air counter 10 may optionally comprise a display 12 for displaying the amount of air passed by the air counter 10. In addition, the air counter 10 may include a module 13, which may include a memory 13m for storing the air counter information and/or other components 13c for relaying this information to an external device. The module 13 may also include a clock function so that the record may be kept as a function of time to enable a physician to calculate the rate of air drainage at different times. Alternatively, the air counter may be preset to record volumes at fixed time intervals and the successive records of volumes may be used to obtain relative rate of air drainage.

In accordance with some embodiments of the invention, the module 13 has the ability to communicate the counting information to an external device, such as a computer or a network. The communication may be via wired or wireless communication. The communication scheme and protocol may be any that is known to one skill in the art.

With an air counter of the invention, a patient will be able to monitor the progress of air drainage. Alternatively, an air counter of the invention may communicate such information to the physician via wired or wireless communication. With this capability, outpatient treatment of a pneumothorax or similar disease becomes a reality.

Many patients with pneumothorax or similar disease do not require admission to the hospital. However, these patients are currently kept in hospitals for observations due to the lack of proper means to monitor the progress of air drainage, and hence there are no criteria to decide which patient can be safely observed at home. Using the air counters of the invention along with a wireless communication technology, information can be continuously monitored from a central location by a healthcare professional while patients are at home. This is the basis for outpatient care of patients with a Pneumothorax. An example of this situation would be a patient that comes in for a CT scan guided lung biopsy by a radiologist. This patient develops a Pneumothorax after the biopsy and has to be admitted for treatment

With our Air Counter technology this patient can be safely treated at the comfort of his home. This will allow marked financial advantage to a medical system that is overburdened with unnecessary expenses.

Some embodiment of the invention relate to air counters having the capability to measure the pressure inside the pleural cavity. If the intra thoracic pressure is high, then a manual or an automatic release button can be triggered allowing for the resolution of this problem. Thus, the safety of using the chest tubes can be improved.

Advantages of embodiments of the invention may include one or more of the following. Currently, clinician has to depend on a pleurovac system with static information to determine when a chest tube should be removed. With dynamic and quantifiable data provided by embodiments of the invention, a clinician can much more accurately determine when to remove the chest tube. This minimizes the possibility of a recurrence of the pneumothorax, which in turn will allow for more efficient care of the patients.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An air counter for use with a chest tube, comprising:
a housing having an inlet and an outlet, at least one of which is configured to connect with the chest tube;
a gas flow detection mechanism; and
an electronic module having a memory for storing gas flow records.

2. The air counter of claim 1, further comprising a display.

3. The air counter of claim 1, wherein the electronic module has a capability to communicate the gas flow records to an outside device.

4. The air counter of claim 3, wherein the capability to communicate is via wireless communication.

5. A method for draining a pleural cavity with a chest tube, comprising:
inserting a chest tube into the pleural cavity; and
connecting an air counter with the chest tube, wherein the air counter comprises:
a housing having an inlet and an outlet, at least one of which is configured to connect with the chest tube;
a gas flow detection mechanism; and
an electronic module having a memory for storing gas flow records.

6. The method of claim 5, further comprising communicating the gas flow records to an outside device.

7. The method of claim 6, wherein the communicating is via wireless communication.
